(19) United States
(12) Patent Application Publication
(10) Pub. No.: US 2009/0093397 A1
(43) Pub. Date: Apr. 9, 2009

(54) PHASEOLUS VULGARIS EXTRACTS, THEIR USE, AND FORMULATIONS CONTAINING THEM

(76) Inventors: Davide Berlanda, Milano (IT); Marco Bertani, Milano (IT); Ezio Bombardelli, Milano (IT); Fabio Donzelli, Milano (IT); Andrea Gardi, Milano (IT); Cesare Ponzone, Milano (IT)

Correspondence Address: MATHEWS, SHEPHERD, MCKAY, & BRUNEAU, P.A., 29 THANET ROAD, SUITE 201, PRINCETON, NJ, 08540, US

(21) Appl. No.: 12/158,070

(22) PCT Filed: Dec. 13, 2006
(86) PCT No.: PCT/EP06/12011

§ 371 Date: Sep. 9, 2008

(30) Foreign Application Priority Data
Dec. 22, 2005 (IT) M12005A 002451

(51) Int. Cl. A61K 38/17; A61P 3/00

(52) U.S. Cl. 514/8

(57) ABSTRACT

Extracts obtainable by extraction from Phaseolus sp. with aqueous solutions, characterised by an α-amylase inhibitor content with an activity equal to or greater than 1,800 USP/mg (HPLC titre equal to or greater than 15% w/w) and a phytohaemagglutinin content of between 1,500 and 6,000 HAU/g, and a process for its preparation.

### SUMMARY OF THE INVENTION

The present invention relates to extracts obtained from the seeds of plants of the genus Phaseolus, and the process for the preparation thereof.

More particularly, the invention relates to extracts of Phaseolus vulgaris seeds, characterised by a content in α-amylase inhibitors and phytohaemagglutinins in established ratios which reduce the absorption of glucose originating from starches in the diet, and reduce the appetite after repeated administration.

### PRIOR ART

α-Amylase inhibitor (αAI) is a glycoprotein contained in the seeds of kidney beans (Phaseolus vulgaris) which inhibits the enzymatic activity of amylase of animal origin, and especially human amylase, in a differentiated, species-dependent way. This inhibitor, which was purified for the first time by Marshall and Lauda in 1974 (J. Biol. Chem., 250 (20), 8030-8037, 1975), has attracted interest because of the effects which its pancreatic amylase inhibiting activity can exert on the intestinal absorption of glucose (deriving from enzymatic hydrolysis of starch), and above all for its potential application in the diet industry. Carbohydrates are an important source of calories and contribute to the synthesis of fats in individuals that are predisposed to obesity or Type II diabetes. In nature, in the evolution of the species, the availability of food for survival was intermittent, so the ability to accumulate energy in excess of the amount required for immediate use was essential. The adipose cells, developed in different parts of the body, are among the sites where energy is accumulated, so that it is easily available when the body needs it. This physiological system, orchestrated by endocrine and neurone secretions, enables humans to survive for long periods, even in the absence of food. However, in the event of abundant food, sedentary lifestyle and genetic reasons associated with the lifestyles of industrialized countries, the system increases uncontrollably the adipose energy deposits with adverse consequences, such as beauty flaw, followed by an overload of the cardiocirculatory system. One of the main problems is obesity, which has reached high levels in some countries, such as the United States of America. Obesity is the primary cause of cardiovascular disease, hypertension and diabetes. Excess weight, which is common among both men and women, causes the subject to eat larger and larger amounts of food, and the result is a deterioration in health. As excess blood glucose leads to an increase in energy deposits, the availability of substances that reduce glucose absorption is very important.

Worldwide demand for anti-obesity substances has led to research and study of foods that counteract the progressive body weight accumulation.

α-Amylase inhibitors have long been identified in different legumes and corn, and specific clinical trials have been conducted in recent years, with mixed results. Depending on the preparation process used for the concentration and isolation of these inhibitors, the results have been contradictory, as many commercial preparations proved to lack effective activity in vivo. According to the first studies of Layer, Carlson and Di Magno (Gastroenterology, 88(6): 1895, 1902, 1985), this problem is apparently due to the high degree of dilution of the inhibitor in highly impure preparations; in fact, preparations of purified inhibitor are proved to be active on α-amylase when are directly introduced into the intestinal lumen.

It has long been known that some seeds and legumes contain substances which, if eaten before they are completely cooked, can be toxic or depress the normal diet. In fact, α-amylase inhibitor is not present in the extracts alone, but is always accompanied in kidney beans by large amounts of phytohaemagglutinins which are considered toxic. The toxicity of phytohaemagglutinins is normally high at the doses in which they are present in nature. Phytohaemagglutinins are glycoproteins like α-amylase inhibitors, and cause hyperplasia, hypertrophy and increase pancreas function at high doses. Phytohaemagglutinins are known to cause enlargement of the pancreas at relatively high doses, thus increasing polyamine accumulation and enzymatic secretion. These glycoproteins survive the intestinal transit and bond to the enterocytes, where they induce secretion of cholecystokinin, a trophic hormone that stimulates pancreatic secretion. Apart from these apparently unfavourable effects on the pancreas and intestine, cholecystokinin also inhibits the appetite, a vital property in the reduction of obesity.

The fragmentary processes described in the literature for the preparation of α-amylase inhibitors involve the extraction with phosphate buffer and the insolubilisation of proteins with ammonium sulphate, and do not provide any selectivity, thus providing extracts that contain high concentrations of phytohaemagglutinins, and must be diluted to obtain extracts with an acceptable level of toxicity. Apart from the biological aspect, known processes include some steps which make difficult to prepare a product that is both active and safe. The problems that arise during extraction with buffers of different ionic strengths and pH are due to the high concentration of protein and polysaccharide contaminants, which make them highly viscous, leading to problems of low filtrability and longer processing times. As these are aqueous extractions, there is also a high risk of microbial contamination of the protein extract, which is difficult to control, especially in the case of highly viscous preparations. All these conditions lead to a loss of product and make difficult to obtain final extracts with a low phytohaemagglutinin content and the corresponding multicomponent standardisation. Various processes have been used to solve the problem of limiting phytohaemagglutinins, including heat treatments, which lead to the breakdown not only of phytohaemagglutinins, but also of α-amylase inhibitors, with the result that the obtained products are scarcely active. In practice, the products on the market have a very low αAI content. Other products which are too highly enriched in α-amylase inhibitors cause unpleasant problems of flatulence when administered in large doses.

### DESCRIPTION OF THE INVENTION

It has surprisingly been found that by preparing products enriched in α-amylase inhibitors having a phytohaemagglutinin content within perfectly tolerable limits, a reduction in body weight proportional to the administered dose can be obtained. The data in rats suggest that the effect on body weight reduction is associated not only with a reduction in the plasma glucose level, but also with a marked reduction in food consumption. Various pharmacological experiments demonstrate that this reduction in food intake, although food is freely available, is not associated with a toxic effect, but with a change in the desire to eat. A long series of experiments conducted first on animals and subsequently on humans have enabled the right balance between the components responsible for the reduction in body weight, without causing undesirable side effects, to be struck.

The products according to the invention are prepared by aqueous extraction and subsequent fractional precipitation with suitable mixtures of ethanol and water.

The process according to the invention uses mixtures of ethanol and water and provides an extract enriched in α-amylase inhibitor whose activity is equal to or higher than 1,800 USP/mg (HPLC titre equal to or greater than 15% w/w) and a phytohaemagglutinin content of between 1,500 and 6,000 HAU/g, so that it can be formulated in products for diet use at sufficiently low doses to obtain the desired result. In addition to this major advantage, the process of the invention produces a significant reduction in the microbe count. Another major advantage is the possibility of obtaining a perfect separation of the main components contained in the starting extract, and consequently an end product highly enriched in inhibitor (αAI), with defined ratios of phytohaemagglutinins.

The process of the invention comprises the extraction of the biomass with buffers having a pH ranging between 3 and 6.5, preferably pH 3.5-5.5, and even more preferably pH 4, at temperatures of between 2 and 25° C., preferably between 4 and 18° C., and subsequent separation of the extract from the biomass by centrifugation.

Suitable buffers for the extraction are typically phosphate, citrate or acetate buffers or dicarboxylic aminoacid buffers, preferably phosphate or citrate buffer.

Depending on the used extractors and on the extraction cycle, 5 to 20 volumes of buffer per part of the starting material are used, preferably 10-12.5 parts, and the mixture is stirred for 1-4 hours, preferably 2 hours; the biomass can be further extracted three more times with a suitable amount of buffer, and in any event until its α-amylase inhibitor and phytohaemagglutinin content is exhausted.

The combined extracts are clarified by filtration or centrifugation and concentrated in vacuum at a temperature of between 25° and 35° C., preferably 30° C., or by ultrafiltration (10,000 Da cut-off) to a volume corresponding to approx. 10% of the weight of the extract after centrifugation.

The next step is a differential precipitation of the concentrated aqueous extract with dilute ethanol, at a final concentration of between 40 and 50% v/v, preferably 45% v/v, operating at a temperature of between 18° and 30° C., and preferably between 20° and 25° C. The precipitate enriched in phytohaemagglutinins is separated, and the filtrate is further diluted with ethanol to an alcohol concentration of 60-70%, preferably 65%.

The obtained precipitate can be centrifuged and/or filtered, redissolved in demineralised water and re-precipitated in 60% ethanol to reduce the saline part. Alternatively, it can be diafiltered through a membrane with a 10,000 Da cut-off. The sediment of the precipitation, which constitutes the extract according to the invention, is dried.

If these processes are used, an extract with the following characteristics can be obtained:
HPLC titre: ≧15% w/w
α-amylase inhibiting activity ≧1,800 USP/mg
haemagglutinating activity: ≧1,500≦6,000 HAU/g

The efficacy of the extracts has been proved in rats, which had free access to food, consisting of a special starch-enriched diet. Wistar rats, individually housed in cages at a constant temperature of 22±2° C. and 60% humidity, were divided into groups of 8-9 animals and treated with a gastric probe for 5 days, at a daily dose in accordance with the following pattern:

1st group: vehicle (0.5% methylcellulose, 4 ml/kg);

2nd group: 300 mg/kg of the extract described in example 2.

Food consumption was recorded immediately after the end of each daily session by weighting the pellets (with an accuracy of 0.1 g). Body weight was recorded once a day immediately before each treatment. Food consumption and weight were recorded throughout the treatment. The statistical analysis was conducted with the ANOVA test.

TABLE 1
Food consumption (grams/animal/day) during the 5 days
of treatment. Mean (with n = 8-9) ± SE

| | 1st | 2nd | 3rd | 4th | 5th |
|---|---|---|---|---|---|
| Control | 23.1 ± 1.6 | 22.9 ± 1.0 | 22.8 ± 1.9 | 22.9 ± 1.9 | 23.0 ± 1.8 |
| | | (−0.86%) | (−1.3%) | (−0.86%) | (−0.43%) |
| Example 2 | 22.1 ± 0.7 | 19.2 ± 0.6* | 20.1 ± 0.9* | 18.6 ± 0.8* | 20.0 ± 1.1 |
| | | (−13.1%) | (−9.04%) | (−15.8%) | (−9.5%) |

Anova split-plot vs. control and day 1 (*p < 0.05); percentages calculated vs. day 1.

During the treatment, the extract described in example 2 reduces food consumption, while water consumption remaines unchanged.

During the 5 days after discontinuance of the treatment, food consumption was uniform and within the normal range (values for the control group in Table 1) for all groups.

Table 2 shows the weight increase data during the treatment.

TABLE 2
Body weight (grams/animal/day) during the 5 days
of treatment. Mean (with n = 8-9) ± SE

| | 1st | 2nd | 3rd | 4th | 5th |
|---|---|---|---|---|---|
| Control | 340.3 ± 4.2 | 350.82 ± 4.3 | 362.4 ± 5.2 | 365.5 ± 4.8 | 369.9 ± 6.1 |
| | | (+3.0%) | (+6.1%) | (+6.9%) | (+8.0%) |
| Example 2 | 341.4 ± 4.1 | 342.08 ± 3.9 | 342.9 ± 4.2* | 342.7 ± 4.5* | 345.2 ± 4.9* |
| | | (+0.2%) | (+0.43%) | (+0.39%) | (+0.95%) |

Anova split-plot vs. control (*p < 0.05); percentages calculated vs. day 1.

The product according to the invention is perfectly tolerated, and can be incorporated in pharmaceutical or dietetic formulations at doses ranging between 100 and 1,000 mg, to be taken at main meals. The extract can be incorporated in drinkable forms or the like, to be taken as appetite suppressants.

The examples below set out illustrate the preparation and the advantages of the invention.

### EXAMPLE 1

### Preparation of a Kidney Bean Extract Enriched in αAI Obtained by Extraction with Phosphate Buffer and Selective Precipitations with Ethanol

A suspension of 490 g of kidney bean flour in 4.9 L of phosphate buffer pH 4.2 was stirred for 1 hour at +22° C.

The suspension was centrifuged and, after clarification of the aqueous centrifugate on paper, concentrated to a weight corresponding to that of the extracted material. The concentrate was diluted with 95% ethanol to a concentration of 45% ethanol to give a precipitate (rich in phytohaemagglutinins and unusable proteins) which was separated by centrifugation at +25° C. and discarded. The centrifuged liquid was further diluted with 95% ethanol to a concentration of 65% to give a precipitate which, after centrifugation and washing with 65% ethanol, was dried under vacuum at a temperature not exceeding 50° C. The obtained product (yield 1.2%) has an α-amylase inhibiting activity of 4200 U/mg, and a haemagglutinating activity of 3500 HAU/g (HPLC titre 35.6% w/w).

### EXAMPLE 2

### Preparation of a Kidney Bean Extract Enriched in αAI Obtained by Extraction with Citrate Buffer and Selective Precipitations with Ethanol

A suspension of 100 g of kidney bean flour in 1.0 L of citric acid 5.75 g/L was stirred for 4 hours at +4° C.

The suspension was centrifuged, and the aqueous centrifugate was concentrated 7.6 times (dry residue, 15.8% w/w). The concentrate was diluted with 95% ethanol to a concentration of 45% ethanol to give a precipitate (rich in phytohaemagglutinins and unusable proteins) which was separated by centrifugation at +25° C. and discarded. The centrifuged liquid was further diluted with 95% ethanol to a concentration of 65% to give a precipitate which, after centrifugation, was dried under vacuum at a temperature not exceeding 50° C. The obtained product (yield 1.59%) has an α-amylase inhibiting activity of 2,200 U/mg, a haemagglutinating activity of 1,800 HAU/g and an HPLC titre of 17.8% w/w.

### EXAMPLE 3

### Preparation of a Kidney Bean Extract Enriched in αAI Obtained by Extraction with Citrate Buffer and Precipitation with Ethanol

A suspension of 120 g of kidney bean flour in 1.2 L of citric acid 5.75 g/L was stirred for 2 hours at +4° C.

The suspension was centrifuged, and the aqueous centrifugate was concentrated 6.8 times (dry residue, 10.0% w/w). The concentrate was diluted with 95% ethanol to a concentration of 45% ethanol to give a precipitate (rich in phytohaemagglutinins and unusable proteins) which was separated by centrifugation at +25° C. and discarded. The centrifuged liquid was further diluted with 95% ethanol to a concentration of 65% to give a precipitate which, after centrifugation, was dried under vacuum at a temperature not exceeding 50° C. The product obtained (yield 0.85%) has an α-amylase inhibiting activity of 3,650 U/mg, a haemagglutinating activity of 1,900 HAU/g and an HPLC titre of 34.3% w/w.

1. Extract obtainable by extraction on Phaseolus sp. with aqueous solutions, characterised by an α-amylase inhibitor content with an activity equal to or greater than 1800 USP/mg (HPLC titre equal to or greater than 15% w/w) and a phytohaemagglutinin content of between 1,500 and 6,000 HAU/g.

2. Process for the preparation of the extract claimed in claim 1, which comprises:
a) Extraction of Phaseolus sp. with aqueous buffers having a pH ranging between 3 and 6.5, and subsequent separation of the extract from the biomass, which can be further extracted with the buffer if required until the α-amylase inhibitors and phytohaemagglutining are exhausted;
b) Filtration or centrifugation of the combined extracts, and concentration to a volume corresponding to approx. 10% of the weight of the extract after centrifugation;
c) Differential precipitation of the concentrated aqueous extract with diluted ethanol, at a final alcohol concentration of between 40 and 50% v/v;
d) Separation of precipitate enriched in phytohaemagglutinins and further dilution of filtrate or centrifugate with ethanol, to an alcohol concentration of 60-70%;
e) Separation of precipitate and re-precipitation from demineralised water with 60% ethanol, or diafiltration through a membrane with a 10,000 Da cut-off, and drying of precipitation residue.

3. Process as claimed in claim 2, wherein phosphate, citrate or acetate buffers or dicarboxylic aminoacid buffers are used.

4. Use of the extracts claimed in claim 1 to prepare appetite suppressants.

5. Compositions containing the extracts claimed in claim 1.
